(19) United States
(12) Patent Application Publication
(10) Pub. No.: US 2007/0100214 A1
(43) Pub. Date: May 3, 2007

(54) Method and apparatus for stimulating exercise

(76) Inventor: John William Steinert, Dix Hills, NY (US)

Correspondence Address: NOLTE NOLTE & HUNTER;CHRISTOPHER B GARVEY, 1077 NORTHERN BLVD, ROSLYN, NY, 11701, US

(21) Appl. No.: 11/373,852

(22) Filed: Mar. 10, 2006

Related U.S. Application Data
(60) Provisional application No. 60/660,319, filed on Mar. 10, 2005.
(60) Provisional application No. 60/704,128, filed on Jul. 29, 2005.

(51) Int. Cl. A61B 5/00; A61H 1/00; A61H 7/00; A61B 13/00; A61B 5/103

(52) U.S. Cl. 600/300; 601/49; 601/86; 600/558; 600/559; 600/595

(57) ABSTRACT

An apparatus for performing multisensory stimulation, to a participant, includes a support platform for supporting the participant. A horizontal linear actuator moves the support platform on a horizontal axis. A vertical linear actuator moves the support platform on a vertical axis. A sensory vestibular input is provided by moving the support platform in two independent axes: a horizontal axis and a vertical axis of control that can be interpolated into any dual axes motion profile of choice.

This application claims priority under 35 USC 119(e) of Provisional Patent Application 60/660,319, filed Mar. 10, 2005 and of Provisional Patent Application 60,704,128 filed Jul. 29, 2005.

### BACKGROUND OF THE INVENTION

1. Field of Invention

The present invention relates to the field of exercise devices, and to a system and method of exercise, which enables the user to exercise both the sensory and motor systems simultaneously in synchronization. The invention is further directed through bio-feedback, physiological monitoring and reassessment procedures to direct the equipment or the operator to make changes in the application of the sensory stimulation variables; those sensory stimulation variables include; vestibular, auditory, visual, tactile, compression, motor and neuro-muscular stimulation.

2. Related Art

See U.S. Pat. No. 6,702,767

Systems have been created for purposes of sensory stimulation but are designed to stimulate variations of the visual, olfactory and auditory systems, (See U.S. Pat. No. 6,702,767).

These systems are for entertainment as well as mood enhancement, relief of stress and in some cases therapeutic applications. The method of application with these systems is mostly designed to create different stimulation environments, these systems do not allow for combined sensory and motor input of vestibular, auditory, visual, tactile, compression, motor and neuro-muscular stimulation to be combined for very specific multi-sensory exercises protocols. Thus there is a need for a multisensory stimulation device that is capable of providing vestibular, auditory, visual, tactile, compression, neuromuscular stimulation and motor stimulation and a method of application of these stimulation variables for purposes of entertainment as well as therapeutic interventions of physical and mental therapy to reduce stress, improve relaxation, and improve multisensory processing, coordination and cognitive capabilities.

### SUMMARY OF THE INVENTION

Brief Description

The present invention is a multisensory stimulation system and method of use of such a system which is designed to overcome the shortcoming of prior art sensory stimulation devices and adapted for flexible, individualized multi-sensory stimulation programs using vestibular, auditory, visual, tactile, compression, motor, neuro-muscular stimulation, bio-feedback and audio-visual entrainment/neurostimulation as exercise variables and a method of application of said variables for development of a multi-sensory stimulation program.

Definitions:

For purposes of this specification:

“System controls” will be defined as a category that will include control and or recording systems that include the visual, auditory, motion, compression, vibration, electrical stimulation, graphic display outputs and physiological response sensors.

“Motor” stimulation will be defined as movement exercise.

“Dual motion” platform refers to the upper motion platform and the lower rotary platform attached to each other

The system includes a dual motion platform that provides vestibular stimulation in a rotary plane around a vertical axis as well as a second upper motion platform that provides a rotary plane motion around a horizontal axis, pure linear motion or a u shaped vestibular stimulation pattern, custom profiles can be combined by programming all three axis in any combination of motion preferred. In some embodiments the rotary motion platform around the vertical axis will be excluded, as well as some of the stimulation variables; tactile, compression, neuro-muscular stimulation. The system has been uniquely designed so that the viewing optical light instrument is fixated to an articulating arm and mounting pole that is attached to the lower rotary motion platform and move as one, as well as the master control system being fixed to the rotary platform and both powered through a commutating ring that allows the operator and participant to be moving together in tandem while allowing access to the control and safety functions of the system manually. The operator is sitting in one of two seating sites that have access to the control panel. The control panel is affixed to an articulating arm and a pole that is mounted to the rotary platform and has a computer monitor, computer system and keyboard attached to said articulating arm as well. The operator is able to swivel the articulating arm and thereby position the control panel, computer system, keyboard and monitor in line of sight viewing.

The viewing optical light instrument is positioned in line of sight of the participant by rotating the light around a rotary axis that is attached to an articulating arm or moving the articulating arm for position. The viewing optical light instrument has led bulbs that are controlled through the primary master control system or the secondary control system. The light is positioned above the participant and is not enclosed in any housing thereby allowing environmental variables to be a factor in the viewing experience. The viewing optical light instrument has circular shapes that have colored lens and a glass diffuser behind them. A mask is optionally applied over the viewing optical light instrument to create different shapes when viewing. The programs that are applied to the light are preprogrammed in the primary master control system or secondary control system. The variable vision stimulation programs vary from visual tracking sequences, color sequences that start at one end of the color spectra and end at the other or participant colors at certain positions in the viewing optical light instrument. The placement of the viewing optical light instrument has an influence and impact on the participant and is capable of being positioned in any position in a horizontal plane above the participant; it is also capable of being tilted down and remain in a parallel line of sight position with the participant in a sitting up position. In addition to the viewing optical light instrument there remains a video monitor that can be placed in line of sight or a video headset that can be placed over their eyes for bio-feedback exercises as well as cognitive and performance exercises.

The support platform is attached to the upper motion platform and contains at minimum 5 transducers located at; the lower base of the spine, upper left shoulder, upper right shoulder, lower left thigh and lower right thigh. This allows individual selection of each transducer to be applied based on operator findings and recommendations; in some embodiments this feature may be excluded. The transducers are powered from an external amplifier and receive an input for the sound jack from the primary master control system, secondary control system or any external audio playback device. Functionally, the transducers are powered through sound files and certain sound files have specified sound files that associate with light programs in such that the sound, light and support experience are integrated and specific for a desired response.

The master control operating system contains a GUI—graphical user interface that allows light and sound programs to be selected and performed on the participant. The sound files are wav files and act as a master time code source for the light programs as well as the motion control when motion control is selected for automatic mode. Once the sound file is selected the output is to headphones, vibrotactile transducers or speakers mounted behind the head of the participant. When in automatic mode, the motion control files are midi files that once selected get converted into analog signals and sent to the motion controller for motion control instruction, the motion control files are considered slave to the sound file. The light programs are midi files and stored in file libraries, once selected through the GUI, they get converted via software code to dmx files and are selected based on the method of application from the operator, then sent to the viewing optical light instrument to power the Led's, they are considered slave files to the master sound file.

The sensory stimulation system has manual control of motion through a mounted HMI- human machine interface that allows independent direction and speed control of the upper and lower dual motion control platform. In addition, the motion controller allows an hourly count log that disables motion from occurring if the account is not up to balance. Additional manual controls exist for the application of compression, neuro- muscular stimulation, audio-visual entrainment/neurostimulation, EEG and biofeedback sensors, these controls are selected based on the assessment and findings as part of the method of application of sensory stimulation from this device.

The present invention therefore discloses a method and device, which provides simultaneous sensory and motor stimulation as an exercise that is further mediated through bio-feedback input, assessment findings and a decision tree based on clinical findings and application of method.

The method of exercise enables a user to exercise the participant's sensory and motor systems, simultaneously with a synchronization process that allows different variables of sensory and motor stimulation and responses to be performed simultaneously with said stimulus, thereby exercising the user's sensory motor system. The synchronization process is performed by a master control system that integrates the position of the device in space and allows control of some of the variables of the device for sensory and motor exercises in synchronization. The synchronization system proceeds to synchronize exercises in several different modes:
1) Operator-user defined instruction set;
2) Preprogrammed exercise protocols; and
3) bio-feedback—this may include manual or automatic response to one or more of the following:
   a) EEG (electroencephalogram)
   b) EMG (electromyogram)
   c) ECG (electrocardiogram)
   d) EOG (electrooculogram),
   e) SCP (slow cortical potentials)
   f) GSR (Galvanic skin response-skin conductance)
   g) Respiration
   h) Pulse oximetery
   i) High resolution temperature
   j) BVP(photoplethysmography)
   k) Vagal tone
   l) HRV(heart rate variability
b. Electroencephalogram (EEG),
c. Skin conductance, and
d. Response times from skills.

The method of exercise provided can be controlled through a primary master control system that allows synchronization of exercise variables. Furthermore, this exercise method will allow for operator, preprogrammed or bio-feedback response driven method application and protocols to be applied. This device will be useful for children and adults that have developmental delays, learning disabilities, brain injuries, degenerative neurological disorders, neurological injuries such as: Stroke, Traumatic Brain Injuries, Autism, Alzheimer's, and Parkinson's, Spinal cord Injury, amputation, entertainment, stress reduction, peak performance training and may enhance sports performance.

### BRIEF DESCRIPTION OF THE DRAWINGS:

FIG. 1 is a side view of the exercise motion platform with all the features assembled of the present invention.

FIG. 1a is horizontal side view of exercise platform with vertical and horizontal axis motion platform mounted to rotary platform with the table top attached

FIG. 1b is an inferior view of the viewing optical light instrument with the LED's numbered 1-25 and the video monitor that can be placed in the line of sight of the participant exercising.

FIG. 2 This perspective shows multiple elevations of just the outer support frame with no inner frame and no linear actuators/screw mechanism attached

2.1 Upper left—top view, 2.2 Lower left-horizontal side view, 2.3 Lower right—horizontal front view

FIG. 2a in this perspective it shows the outer frame no inner frame - top view showing lower pulley system mounted on bottom of frame rails, this lower pulley system (5) attaches to and drives the linear actuator/screw mechanism in the vertical axis.

FIG. 2b in this perspective this shows a horizontal side view with the vertical linear actuator/screw mechanism (6) installed and attached to the lower pulley system (7) that drives it.

FIG. 3 in this perspective this shows an outer frame view (50)—and inner frame (9)—with linear actuators/screw mechanism attached. 3.1 Upper left—top view, 3.2 Lower left—horizontal side view, 3.3 Lower right—horizontal front view

FIG. 3a outer frame (50) and inner frame (9)—top view Showing horizontal linear glide rails (8) mounted on top of inner frame (9), and the horizontal mounting plate (10) is attached to the horizontal linear glides (8) and the horizontal linear actuator/screw mechanism (11)

FIG. 3b This perspective shows the outer frame (50) and inner frame (9)—horizontal side view Showing the horizontal mounting plate (10) is attached to the horizontal linear actuator/screw mechanism (11) and the upper pulley system (12) is attached to a motor shaft, the motor is attached to the horizontal axis motor mount (13)

FIG. 3c This perspective shows the outer frame (50) and inner frame (9)—horizontal front view Showing the horizontal mounting plate (10) is attached to the horizontal linear glide rails (8) and the upper pulley system (12), and the vertical axis linear actuator/screw mechanism (6).

FIG. 3d This perspective shows the outer frame (50) and inner frame (9)—horizontal side view Showing the horizontal axis motor (14) is attached to the upper pulley system (12), horizontal axis motor mount (13) and the vertical axis motor (15) attached to the vertical axis motor mount (16) and attached to the vertical axis lower pulley system (5) and drives the vertical axis linear actuator and screw mechanism (6).

FIG. 3e This perspective shows outer frame (50) and inner frame (9)—horizontal frontal view Showing the horizontal axis motor (14) is attached to the upper motor mount (13) and the upper pulley system (12), and the inner frame (9) is attached to the outer frame (50) with vertical linear glide rails and bearing assembly (17).

FIG. 4a This perspective shows the entire motion platform that controls the two axis of vertical and horizontal motion that can be used as a stand alone motion device as seen in 4a.

FIG. 4b This perspective shows the upper two axis assembly as seen in 4a mounted to the rotary platform as shown below in 4b.

FIG. 5 is a horizontal view of rotary table has two mounting frames, an upper frame (18) and a lower frame (19) above and below a center hub (20) that allows rotation around a center axis. Attached to the upper frame (18) are 6 wheels that help support the weight placed on top of it. In addition the rotary base has a commutating ring (21) that allows electrical current to be run through it.

Attached to the upper rotary base platform is a motor and rotary gear drive assembly (22) that is attached to a chain assembly (23) that allows the rotary base to rotate around a center axis. The upper frame assembly (18) will rotate and the lower platform (19) remains fixed: therefore the motor and gear drive assembly (22) will rotate with the upper platform (18).

FIG. 6 illustrates the light control schematic for manual mode.

FIG. 7 illustrates the light, sound and motion control schematic in automatic mode.

FIG. 8 illustrates light, sound and motion control schematic in physiological response mode.

FIG. 9 illustrates the manual motion control input.

FIG. 10 illustrates the automatic motion control schematic.

FIG. 11 illustrates the automatic motion control schematic using physiologic response mode

FIG. 12 illustrates the vasopneumatic pump/compression device.

FIG. 13 Illustrates the Transcutaneous Electrical Nerve Stimulation (TENS)/neuromuscular stimulation:this will provide current to electrodes that will be placed on the clients extremities.

FIG. 14 lists the physiologic response sensors that may be incorporated as a physiologic response input.

FIG. 15 illustrates the support platform in an overhead perspective, the client would be lying down on the table and the transducers would line up with the shoulder region on each side, the lower spine and each leg, the in table speakers are displayed where the head would be positioned.

FIG. 16 displays the video headset that can be placed over the users eyes.

FIG. 17 illustrates the decision tree that describes implementation of the sensory stimulation variables that will be used during the exercise programs.

### DESCRIPTION OF SENSORY STIMULATION DEVICE

Turning now to the drawings:

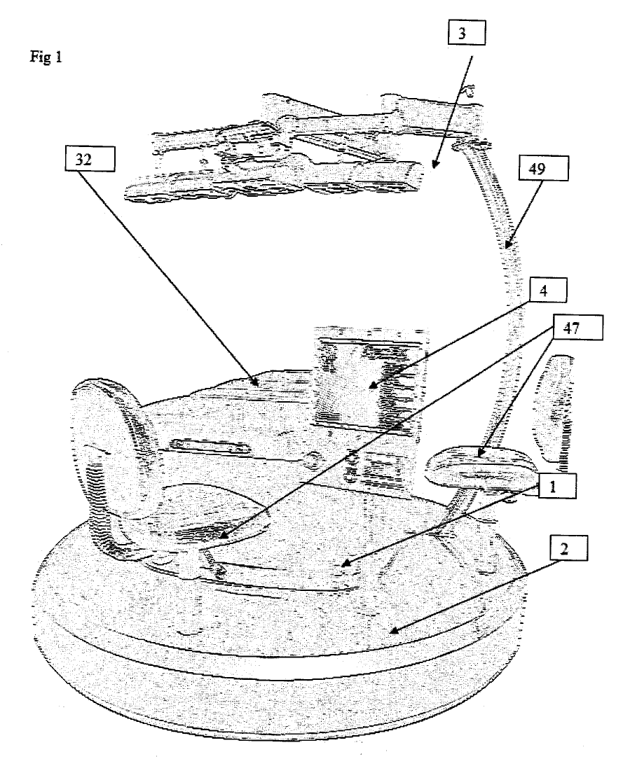

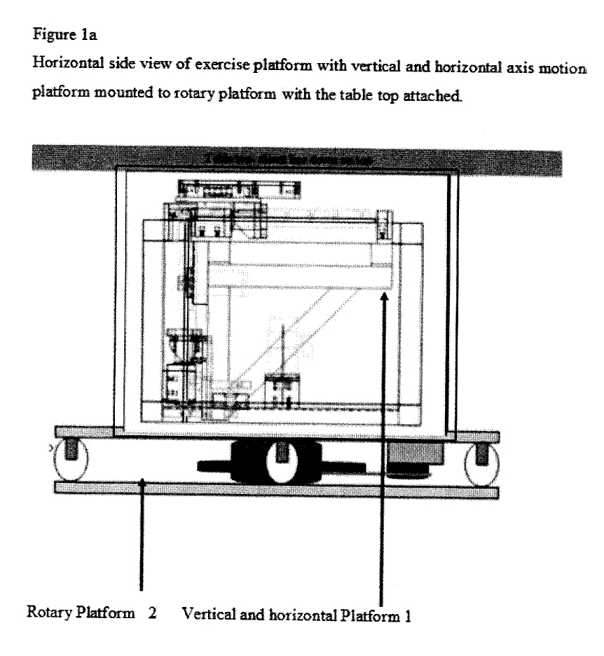

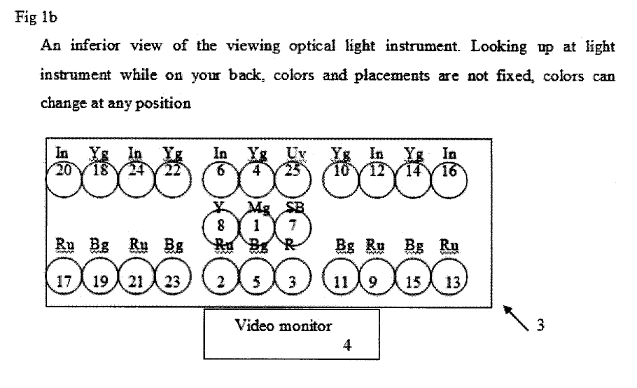

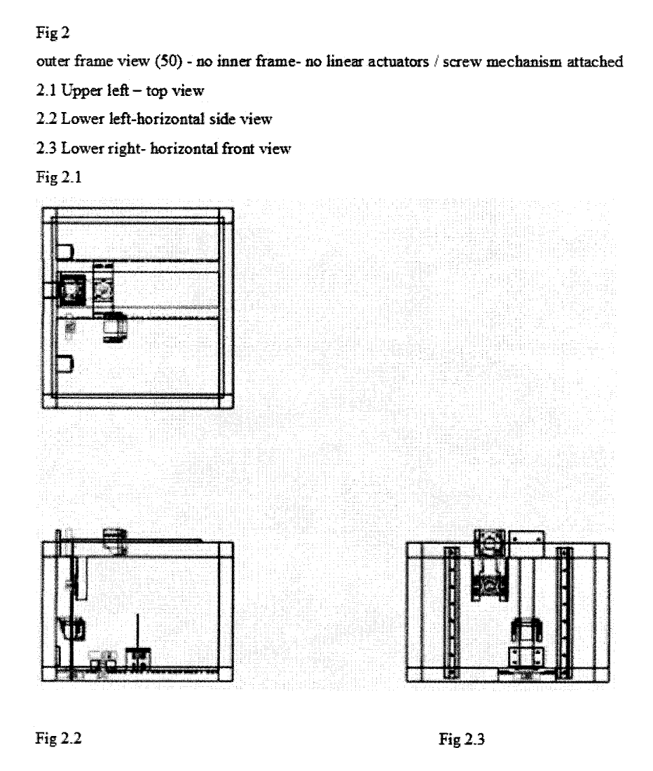

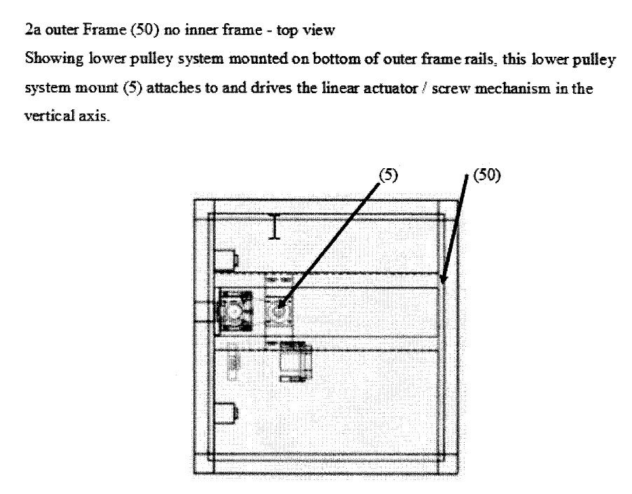

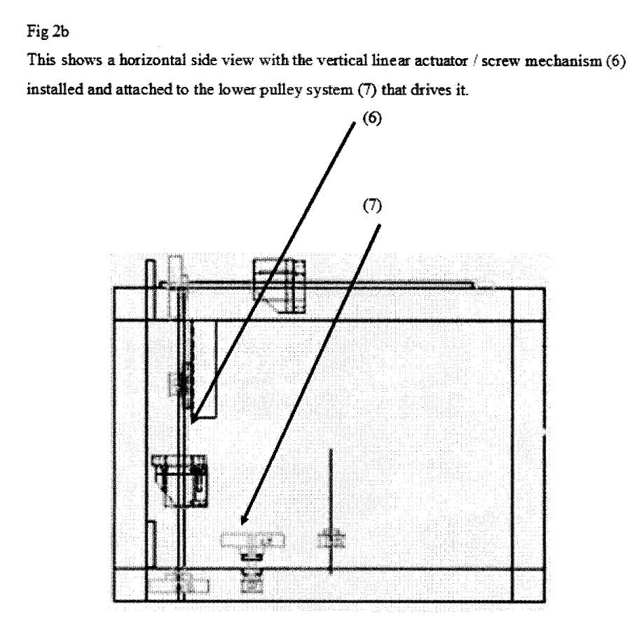

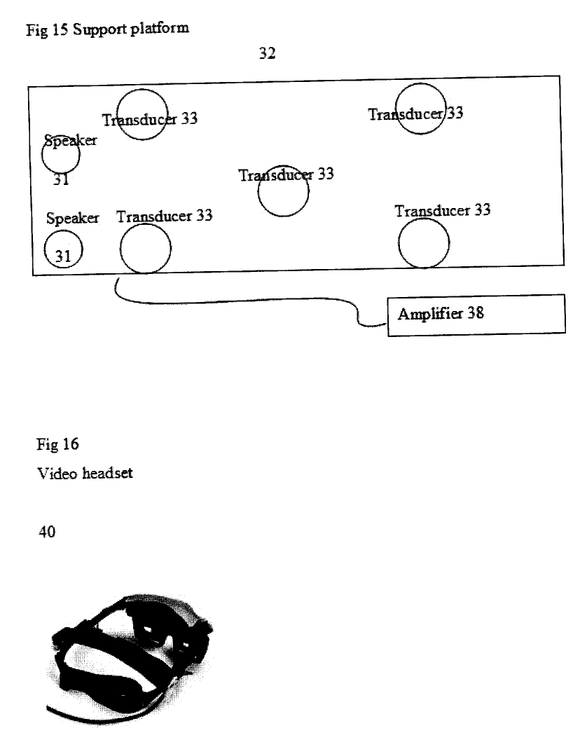

FIG. 1.2 shows a side view of the exercise device. A person will be positioned on the support platform (FIG. 15) 32 and will be positioned in a sitting or lying position. The table 32 is capable of be positioned for upright sitting to full supine (flat) positioning. The viewing optical light instrument (FIG. 1b) 3 is attached to the articulating arm assembly 48 which is attached to the mounting pole 49 which is attached to the upper rotary mounting frame (FIG. 5) 18. The viewing optical light instrument 3 will be positioned above the person's head and in line of sight. The viewing optical light instrument 3 will provide different colored stimulus at different bulbs in the light instrument, this is determined by the operator.

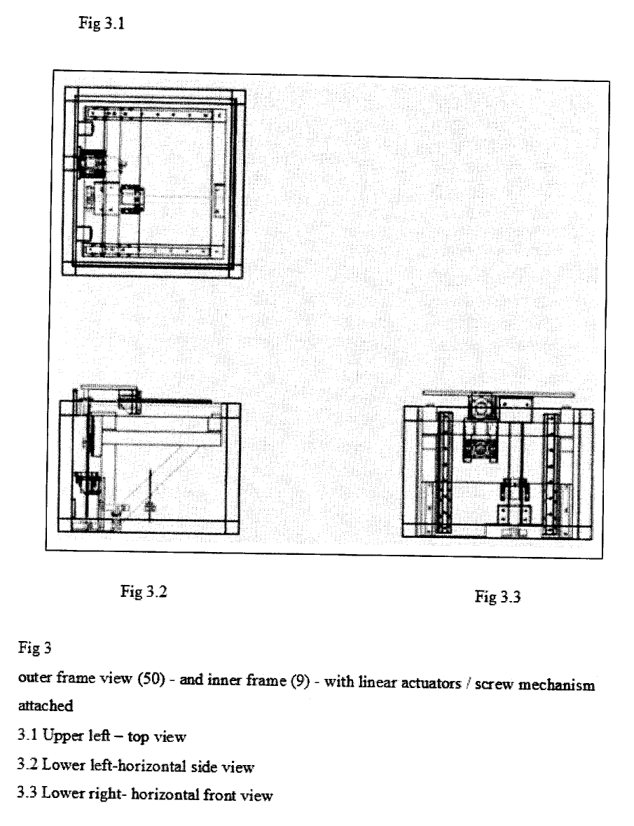

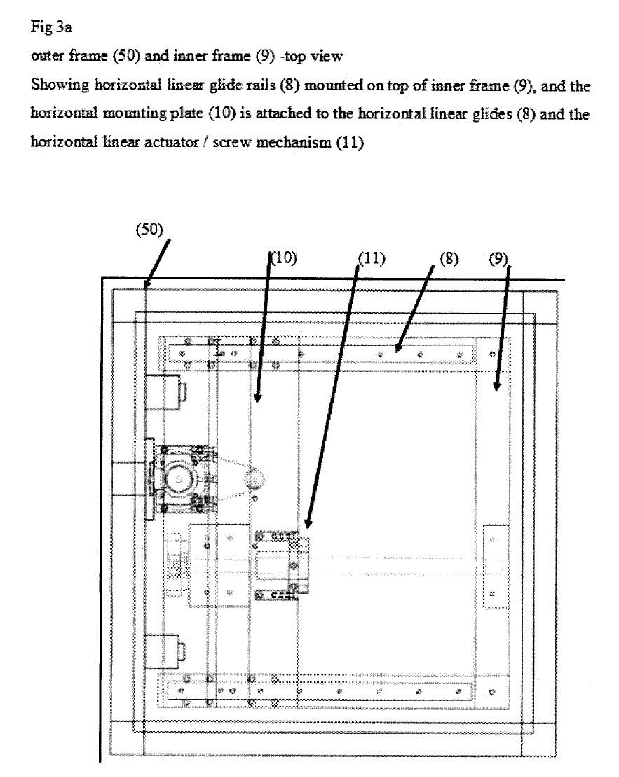

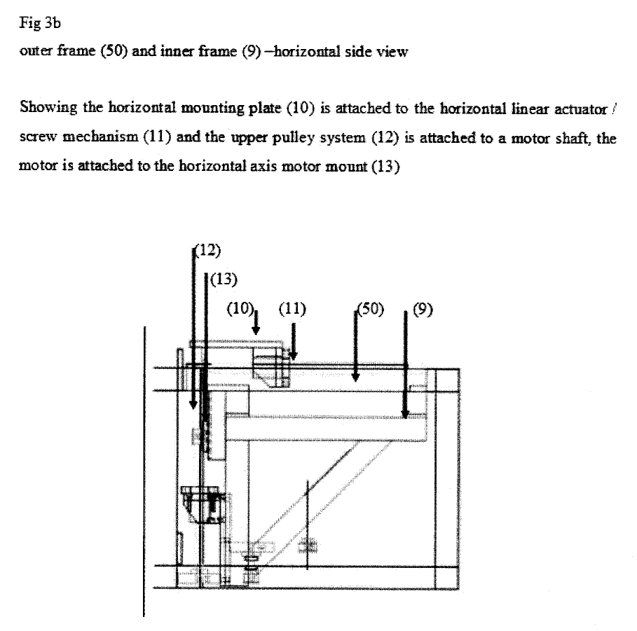

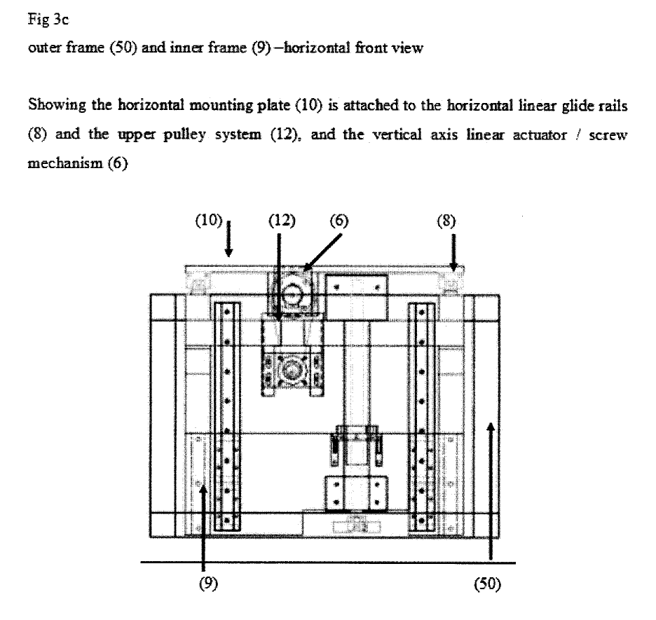

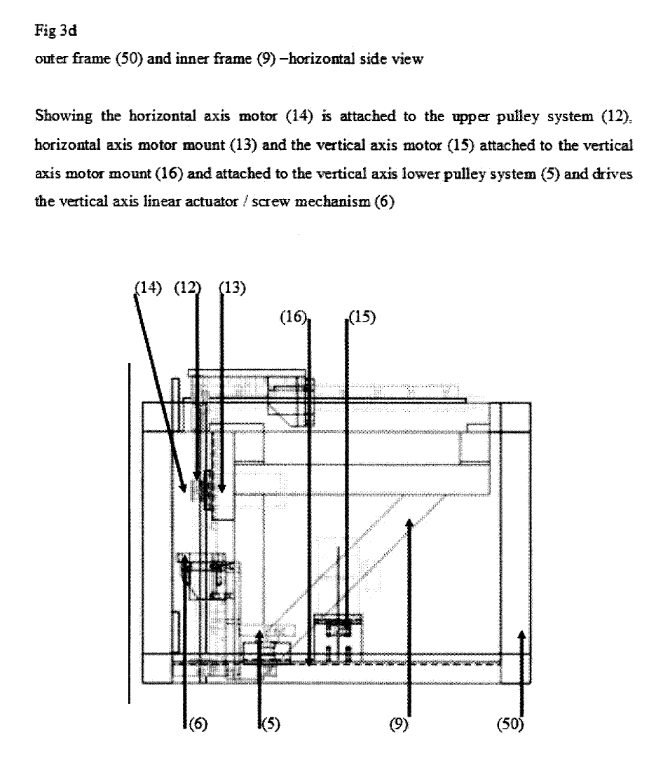

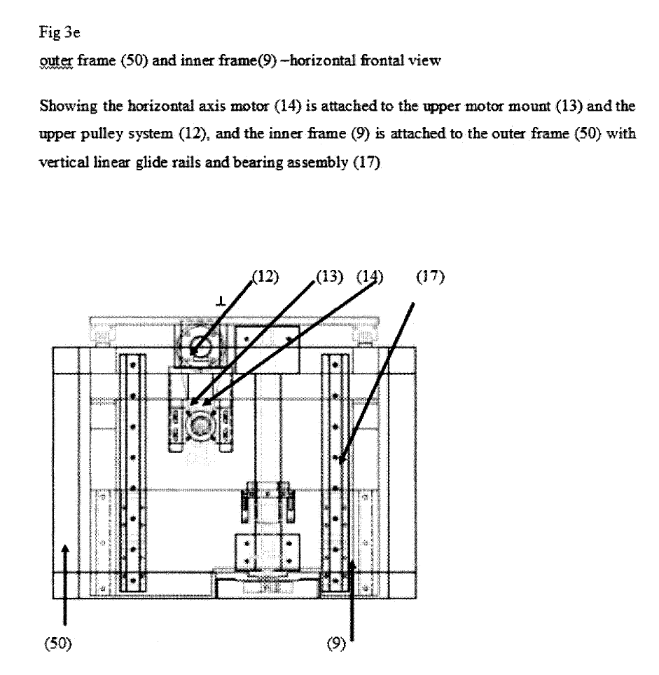

The support platform 32 will be mounted to the horizontal mounting plate (FIG. 3a) 10. The horizontal mounting plate 10 is attached to the inner frame assembly (FIG. 3a) (9) by way of linear glides (FIG. 3a) 8 and the horizontal linear actuator/screw mechanism (FIG. 3a) 11. This attachment allows the table to move in a linear fashion in a horizontal axis. Furthermore, the support platform 32 is capable of rotating its position on the horizontal mounting plate 10 where in the table can be moving in a left to right linear motion or a head to toe linear motion based on the support platform's 32 position relative to the horizontal linear actuator/screw mechanism. As Shown in FIG. 3b the upper pulley system 12 is attached to the horizontal linear actuator/screw mechanism 11 and provided the motion for linear movement in the upper motion platform. In FIG. 3c a horizontal front view is shown displaying the details of the upper pulley system 12 and the linear glide rails 8.

The support platform 32 also provides a vertical plane component of motion. This is seen in FIG. 3d; the vertical axis motor 15 is attached to the vertical axis motor mount 16 and drives the vertical axis lower pulley system 5 which moves the vertical axis linear actuator/screw mechanism 6 up and down. The vertical axis linear actuator 6 is attached to the inner frame 9 with vertical axis linear glide rail (FIG. 3e) 17. As the operator selects different motion profiles from the motion controller FIG. 9 the horizontal linear actuator/screw mechanism 11 and the vertical linear actuator/screw mechanism 6 are controlled and create different motion profiles such as but not limited to: circular around a horizontal axis, U shaped, pure linear, pure vertical and sinusoidal.

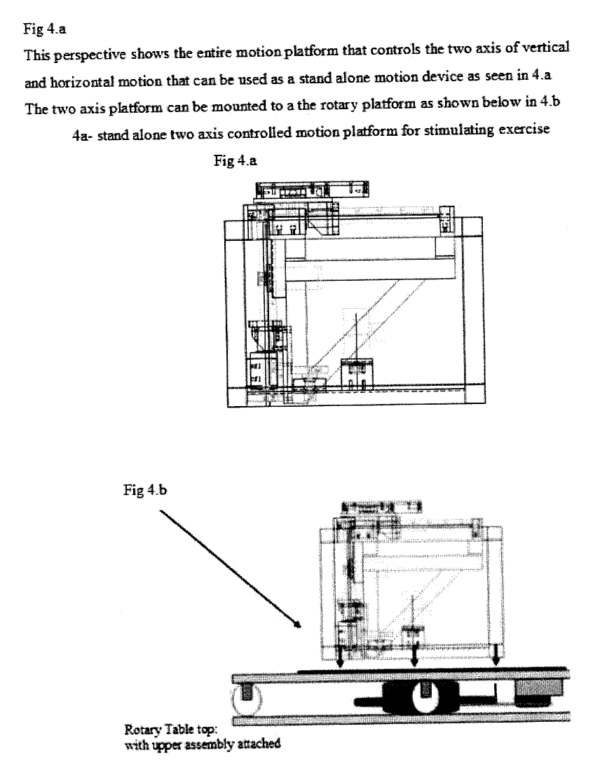

As Shown in FIG. 4a the two axis platform can be used as a stand alone motion platform in some embodiments. In FIG. 4b the two axis motion platform is mounted on top of the rotary platform (FIG. 5) 2. FIG. 5 shows the rotary platform 2 with two mounting frames the upper frame 18 and the lower frame 19 attached to a center hub assembly 20. Attached to the upper frame 18 are six wheels that help support the weight placed on top of it. The rotary base 2 has a commutating ring 21 mounted inside the center hub 20 that allows power to be run to all system controls of the device. Attached to the upper rotary base platform 18 is a motor and rotary gear drive assembly (22) that is attached to a chain assembly (23) that allows the upper rotary base 18 to rotate around a center axis. The upper frame assembly (18) will rotate and the lower platform (19) remains fixed: therefore the motor and gear drive assembly (22) will rotate with the upper platform (18). As seen in FIG. 4b when the vertical and horizontal axis platform 1 is attached to the rotary platform 2 there are many different combination of motion that can be applied to the participant while on the support platform 32.

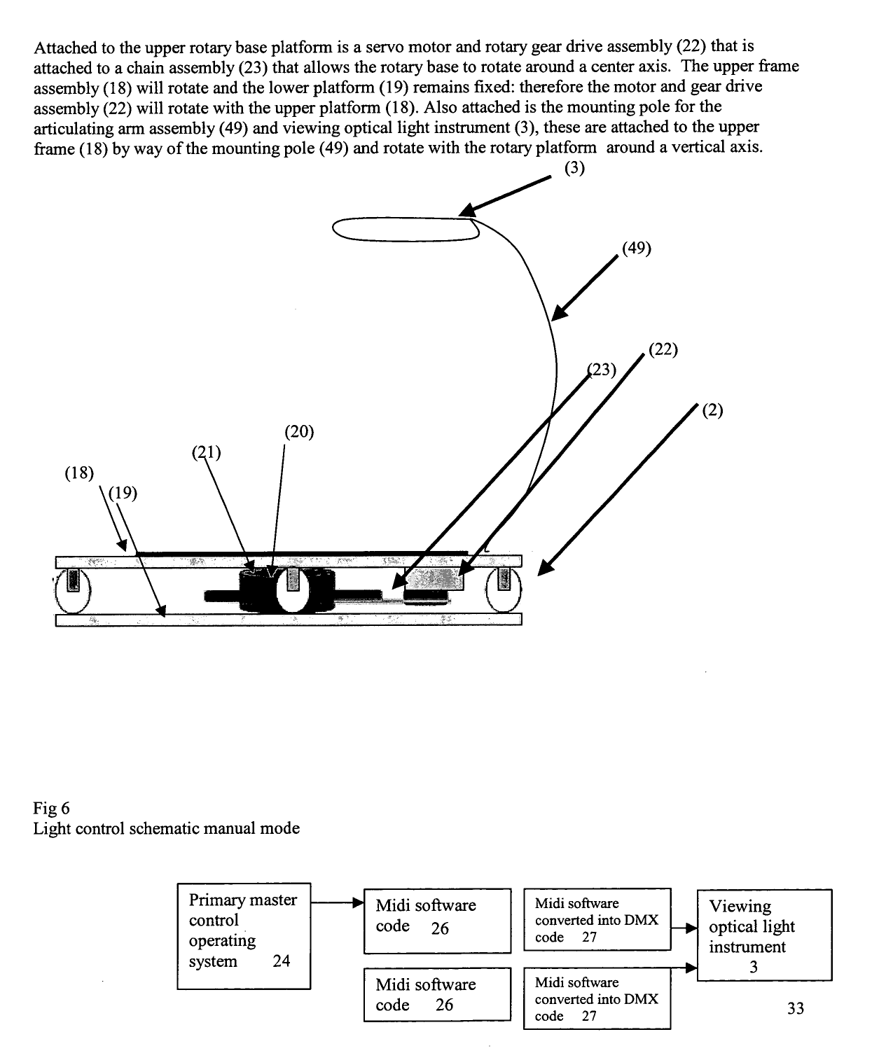

Now turning to the control system as seen in FIG. 6, the light control in manual mode uses the primary control operating system 24 which controls midi software code 26 that is converted into dmx code 27 and then sent to the viewing optical light instrument 3. The primary master control system 24 uses a GUI touch screen interface (FIG. 18) 39 that allows selections of the midi software code for the light programs. The secondary control operating system 25 provides for an alternate method to select and run a light program that is contained in the midi software code 26.

FIG. 7 shows the light, sound and motion being controlled when selected in automatic mode. The primary master control system 24 sends midi software code 26 to be converted into dmx 27 and then sent to the optical viewing light instrument 3, the vertical and horizontal, and rotary motion platform 1,2. In addition the primary master control system 24 sends a sound source wav file 28 or similar equivalent that acts as a master timeline source code 35 for the viewing optical light instrument 3 and the vertical and horizontal motion platform 1 and the rotary platform 2 as well as provide audio output to headphones30, Speakers31, support platform 32 and transducers 33.

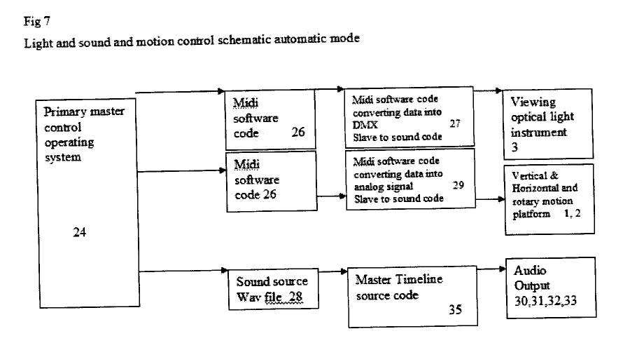

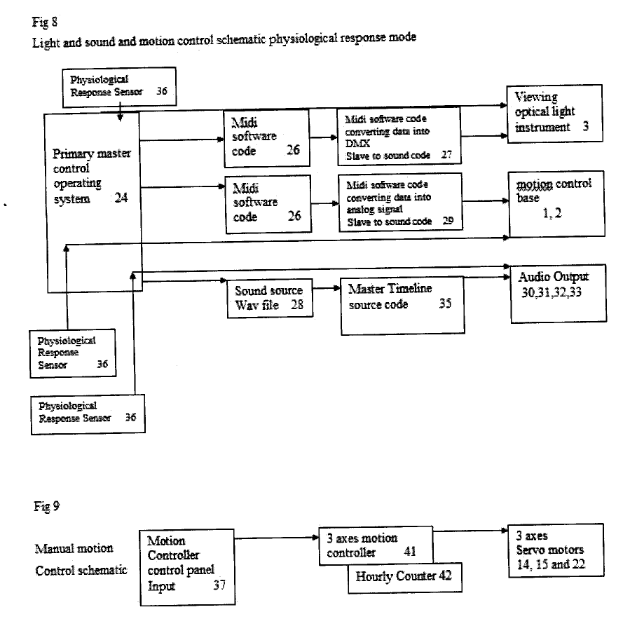

FIG. 8 shows the primary master control system 24 coordinating the physiological response sensor 36 output and directing it directly to the viewing optical light instrument 3, directly to the motion control bases 1 or 2, directly to an audio output device, headphones30, Speakers3 1, support platform 32 and transducers 33. FIG. 9 shows the manual motion control input 37 that will control the 3 axes motion controller 41 and control the hourly counter 42 as well, furthermore the signal is directed to the vertical axis servo motor 15 and horizontal axis servo motor 14 and rotary servo motor 22 of the motion platform. FIG. 10 shows how the automatic motion control would work, by taking the signal from the primary master control platform 24 or the secondary control operating system 25, the signal would be sent into the analog/digital input of the 3 axes motion controller 43 and then control the servo motors; vertical axis servo motor 15 and horizontal axis servo motor 14 and rotary servo motor 22 of the motion platform. FIG. 11 shows how the motion control would be affected by the physiologic sensor 36 that sends a signal to the secondary operating system 25 and the primary master control operating system 24 that sends it's signal to the analog/digital input on the 3 axes controller that then controls the vertical axis servo motor 15 and horizontal axis servo motor 14 and rotary servo motor 22 of the motion platform.

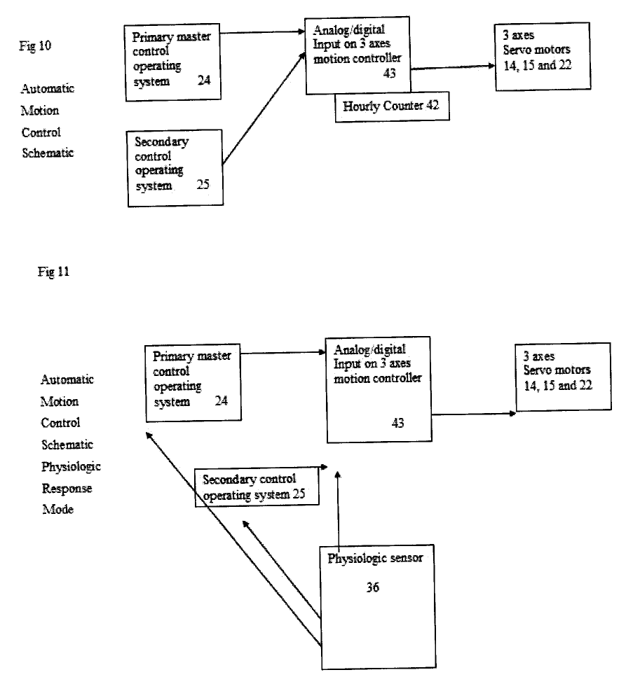

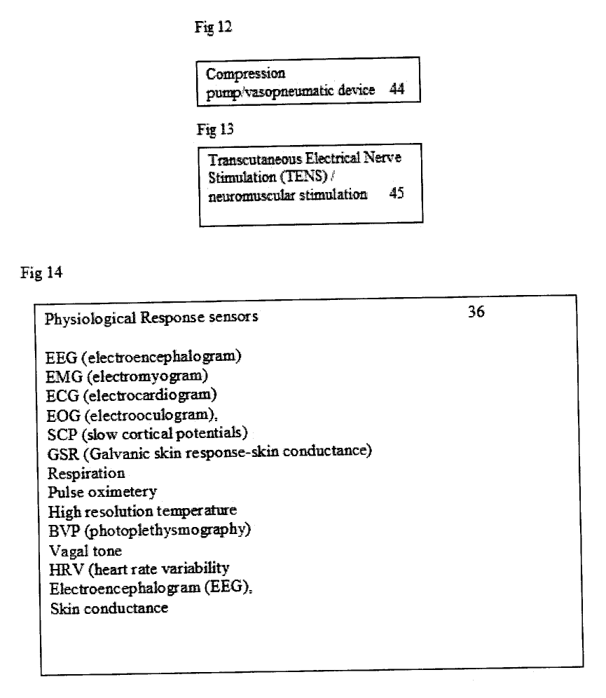

FIG. 12 represents the compression pump/vasopneumatic device 44 that will be placed on a participant's extremity based on the operators selected preferences. FIG. 13 represents the Tens/neuromuscular stimulation unit 45, this will be providing current to electrodes that will be placed on the client's extremities. FIG. 14 represents physiological response sensors 36 that may be used as objective variables for operator program modification or automatic control of system controls.

FIG. 15 shows a top view perspective of the support platform 32 with the five transducers 33 in place and the two speakers 31 in place at the head of the table. The transducers 33 are powered by an amplifier 38 and are individually controlled and can be coordinated to be turned on and off in any combination. The center transducer always stays on. The speakers 31 receive input from the primary master control system 24 or the amplifier 38 or any third party audio output device.

FIG. 16 represents a video headset that can be worn by the participant while they are on the motion platform and perform different types of bio-feedback exercises as well as cognitive exercises.

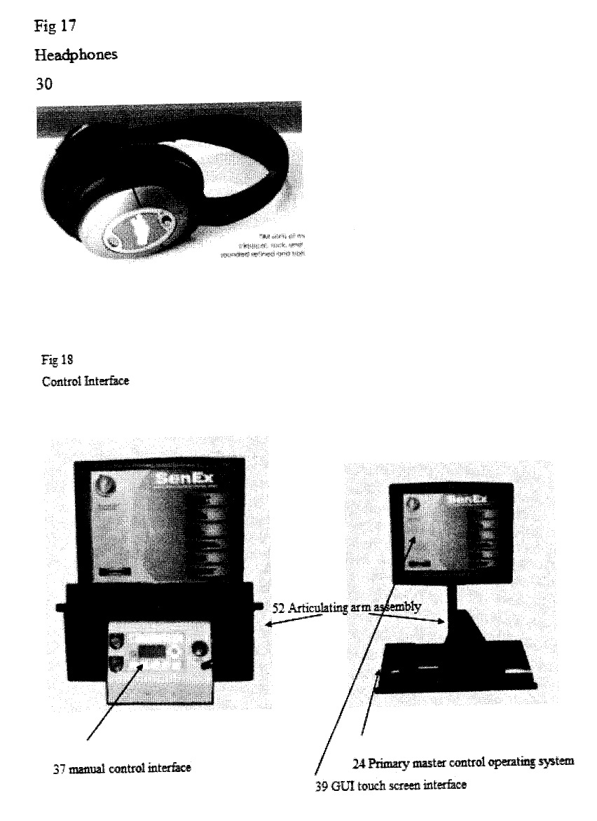

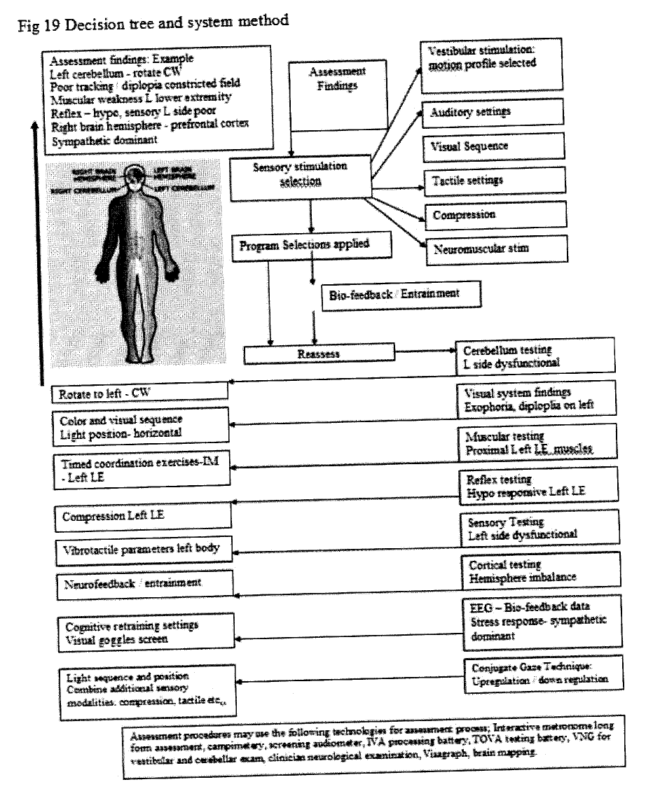

FIG. 17 represents headphones that will receive input from the primary master control system 24, the secondary master control system 25 or any third party audio output device.

FIG. 18 represents the manual control interface 37 and articulating arm 52 that mounts the GUI touch screen interface 39 and primary master control operating system 24. The manual control interface 37 contains a power on, power off and emergency stop button. All participant programming done through the primary master control interface 24 and manual control interface 37, unless the secondary control operating system 25 is selected.

FIG. 19 represents the decision tree and system method 46 in an outline form and by way of example how the controls and selection criteria are made for the variables of the multisensory training system.

I claim:

1. Apparatus for performing multisensory stimulation, to a participant, said apparatus compromising:
a support platform for supporting the participant;
a horizontal linear actuator for moving the support platform on a horizontal axis;
a vertical linear actuator for moving the support platform on a vertical axis;
whereby sensory vestibular input is provided by moving the support platform in two independent axes; a horizontal axis and a vertical axis of control that can be interpolated into any dual axes motion profile of choice.

2. Apparatus for performing multisensory stimulation, to a participant, by a plurality of sensory inputs, said apparatus compromising:
a rotary motion platform;
a viewing optical light instrument affixed to an articulating arm;
which articulating arm is attached to a mounting pole;
which mounting pole is attached to the rotary motion platform;
whereby the viewing optical light instrument remains in a fixed position relative to the rotary motion platform;
thereby allowing visual input to remain stationary relative to the rotary motion platform while the platform rotates.

3. Apparatus according to claim 2, further compromising:
a support platform for supporting the participant;
a horizontal linear actuator for moving the support platform on a horizontal axis;
a vertical linear actuator for moving the support platform on a vertical axis;
whereby sensory vestibular input is provided by moving the support platform in two independent axes; a horizontal axis and a vertical axis of control that can be interpolated into any dual axes motion profile of choice;
said apparatus controlled by a motion controller with analog/digital input and output capability and a motion control interface that allows independent control of all 3 axes of motion.

4. The multisensory apparatus according to claim 3:
in which the support platform is position adjustable;
in which the viewing optical light instrument has a housing with circular and geometric shaped viewing ports and the viewing optical light instrument is positioned on the articulating arm, with a rotating axis above the support platform platform;
in which the plurality of sensory inputs, comprise:
the rotary motion platform,
the viewing optical light instrument,
the horizontal linear actuator,
the vertical linear actuator,
an auditory stimulus provided through a headphone set, a set of externally mounted speakers,
or through support table transducers,
a vasopneumatic device for providing joint or muscle compression to any or all of the extremities, said vasopneumatic device can simultaneously provide compression, heat or ice, and
a neuromuscular stimulator, which may be applied to any or all of the extremities;
an automatic controller of the sensory inputs, said automatic controller responsive to sensor based bio-feedback responses; and
a manual controller of the sensory inputs, with a manual computer program selection interface; and
means for audio-visual entrainment comprising either:
glasses with Led lights that flicker, for placement on the participant; or
said viewing light optical instrument;
which receives input from an entrainment processor; and
a means for visual information to be applied with a visual headset that project a large screen image wherein the participant does not see their peripheral environment while said visual headset device is operating; and
a means for integrating and mounting a software based timed coordination repetition exercise device using auditory stimulus through headphones or speakers and optional visual feedback; and
a means for a primary master control operating system that incorporates a touch screen interface with pre-programmed parameters for program selection; and
a means for a secondary control operating system with advanced research programming flexibility; and
a means for a decision making method and program parameter selection based on a clinical testing decision tree specific to said multisensory stimulation apparatus and sensory input variables.

5. The motion controller as defined in claim 3, further comprising an automatic control mode that allows analog/digital control of all 3 axes of the dual motion platform.

6. The motion controller as defined in claim 5, in which the automatic mode includes an analog/digital interface which interfaces with a master control system, and the master control system provides a midi output signal to control the motion control of the dual motion platform.

7. The motion controller as defined in claim 5 is integrated into a bio-feedback module through analog/digital sensor based input into the motion controller, wherein signals sent to the motion controller are calculated in a software code in the motion controller and make adjustments to the speed of the dual motion platform based on the biofeedback signals received from the biofeedback module.

8. The multisensory stimulation system according to claim 4, wherein the support platform is adjustable at three hinged positions:
two positions for the lower extremities, and
one position for the upper torso allowing the participant to be positioned in:
sitting,
supine,
legs elevated,
side lying, and
prone positions, to receive multisensory stimulation.

9. The support platform of claim 8, wherein multiple lying and seating positions are performed in a rotating mechanism that allows the support platform to be rotated, and locked in a 90 degree range along a horizontal axis, and said support platform has a sound transducer in multiple positions to provide specific targeted tactile sensory input.

10. The support platform of claim 9, further compromising a external amplifier that is mounted to the multisensory system and controlled through the primary master control system sound source code or through a externally mounted audio playback device to include, CD, DVD, MP3, hard disk, flash memory or equivalent audio play back device that will send an audio signal to vibrotactile transducers that were selected to be operational on the external amplifier, and the transducer selection options are as follow: based on a participant lying supine—facing up—on the support platform include: the upper left, upper right, sacral, lower left, lower right or any combination thereof, using no less than 5 transducers positioned in above said selection options.

11. The multisensory stimulation system according to claim 3, in which the viewing optical light instrument allows adjustment of plane of viewing from horizontal to vertical in a graded manner, said viewing optical instrument is attached to the lower rotary axes of said motion platform and is capable of remaining in a fixed position while single or multiple axes vestibular stimulation is provided, said viewing optical light instrument rotates with said lower rotary motion platform.

12. The multisensory stimulation system according to claim 3, wherein said viewing optical light instrument further compromises 25 viewing ports with different light spectra/colors, dimming rates and flicker rates to be selected, programmed and viewed while on the support platform.

13. The viewing optical light instrument as defined in claim 3 comprises led bulbs that are housed in a rubber tubular material and compression fit at one end, then the rubber tubular material is inserted into a machined plate that houses a clear glass lens, a colored lens, a white glass diffuser, a circular machined recess allows the rubber tubular material to be compression fitted into said plate, these 25 rubber tubular material led fixtures are then fastened down with a holding panel and the led bulbs are strategically placed in an array of 25 bulbs and are controlled with the master control operating system through preprogrammed selections or though a secondary control operating system through customized settings and software interface.

14. The viewing optical light instrument as defined in claim 3, provides visual stimulation programs, which programs stay positioned in the central visual field on the client, the viewing optical light instrument is positioned in front of the client and will be positioned based on the support platform position.

15. The viewing optical light instrument as defined in claim 3 provides visual stimulation programs compromising visual tracking, vor rotary programs, directional attack and cognitive skill building wherein the visual stimulus of a preprogrammed specific color spectra, and flash rate will move from left to right or right to left, up and down or down and up, or diagonal left and right or right and left in all 4 quadrants, the lights will move in preprogrammed sequences the entire length of the viewing instrument or in limited ranges based on the leds that are selected and the viewing optical light instrument is capable of being rotated in position for directional preference in visual tracking as well as being positioned anywhere in a horizontal plane with the client on there stomach or back, or a vertical plane with the client sitting upright or any placement in between.

16. The viewing optical light instrument as defined in claim 3 compromising a led lighting devices that is addressed with their own computer network address, this unique address allows said led to be programmed with infinite lighting effects and performance features by the primary master control operating system or the secondary control operating system.

17. The viewing optical light instrument as defined in claim 15 wherein the led light program selection is defined for client performance objectives is controlled through the primary master control system using a midi and a dmx software code, the software code instruction from the primary master control system allows for dimming effects, flash effects, color changes, led position changes and timed programming sequencing of said led device.

18. The software code as defined in claim 17 from the primary master control system compromising a touch screen interface uses a table file for allocating, storing and retrieving files for selection for led lighting effects, led position changes and timed programming sequencing of said led device.

19. The multisensory stimulation system according to claim 3 compromising a auditory stimulus means that is controlled through the primary master control operating system provides input to headphones, support table, external speakers or any combination thereof.

20. The auditory stimulus means of claim 19 further compromising the primary master control operating system input to selectively filter frequencies of the left and right side channel independently, select the modulation type applied to the sound and select the balance of the left and right side channels.

21. The auditory stimulus means of claim 20 further compromising the secondary control operating system input to selectively filter frequencies of the left and right side channel independently, select the modulation type applied to the sound and select the balance of the left and right side channels.

22. The secondary control operating system as defined in claim 3 may run simultaneously on the primary master control operating system and include a means of control for the viewing optical light instrument Led's, the dual motion platform, the support platform, and the auditory stimulus.

23. The secondary control operating system as defined in claim 3 may operate on a second computer control system mounted to the multisensory stimulation apparatus and include a means of control of the viewing optical light instrument Led's, the dual motion platform, the support platform, and the auditory stimulus.

24. The multisensory stimulation apparatus of claim 3 comprising a means of compression with a device using an externally mounted vasopneumatic pump with heat or cold as a selection variable.

25. The multisensory stimulation apparatus of claim 3 comprising a means of control for neuromuscular stimulation to selected anatomical area through the primary master control operating system and the secondary operating control system.

26. The multisensory stimulation apparatus of claim 3 comprising a means of manual control for neuromuscular stimulation to selected anatomical area.

27. The multisensory stimulation apparatus of claim 3 comprising a means of bio-feedback control from at least one bio-feedback sensor input to the motion controller and the motion control of 3 axes may independently or simultaneously be controlled through the bio-feedback sensor input.

28. The multisensory stimulation apparatus of claim 3 compromising a means of bio-feedback control using a sensor which is selected from a group of sensors of electrophysical signals including; EEG (electroencephalogram), EMG (electromyogram), ECG (electrocardiogram), EOG (electrooculogram), SCP (slow cortical potentials), GSR (Galvanic skin response-skin conductance), Respiration, Pulse oximetery, high resolution temperature, BVP (photoplethysmography), vagal tone, and HRV (heart rate variability) that will allow electrophysiological feedback and manual control of said multisensory stimulation apparatus sensory inputs by means of motion control input interface, the primary master control operating system and the secondary control operating system.

29. The multisensory stimulation apparatus of claim 3 compromising a means of bio-feedback control using a sensor which is selected from a group of sensors of electrophysical signals including; EEG (electroencephalogram), EMG (electromyogram), ECG (electrocardiogram), EOG (electrooculogram), SCP (slow cortical potentials), GSR (Galvanic skin response-skin conductance), Respiration, Pulse oximetery, high resolution temperature, BVP (photoplethysmography), vagal tone, and HRV (heart rate variability) that will allow electrophysiological feedback and automatic control of the motion controller and 3 axes of motion.

30. The multisensory stimulation apparatus of claim 3 compromising a means of a audio-visual entrainment stimulation that is applied with clear, colored or opaque glasses and sound input through the headphone jack, the entrainment system will be monitored by means of bio-feedback control unit and allow for manual control of said multisensory stimulation inputs by means of motion control input interface, the primary master control operating system and the secondary control operating system.

31. The multisensory stimulation apparatus of claim 3 compromising a means of a audio-visual entrainment stimulation that is applied with clear, colored or opaque glasses and sound input through the headphone jack, the entrainment system will be monitored by means of bio-feedback control unit and allow by means of bio-feedback control, electrophysiological feedback that will provide input to the motion controller for 3 axes of motion control and instruction.

32. The multisensory stimulation apparatus of claim 3 compromising a means of audio-visual entrainment stimulation that is applied by means of input to the viewing optical light instrument and sound input through the headphone jack, the entrainment system will be monitored by means of bio-feedback control unit and allow for manual control of said multisensory stimulation apparatus sensory stimulation inputs by means of motion control input interface, the primary master control operating system and the secondary control operating system.

33. A method of providing multisensory stimulation to a participant using a system including steps of: assessing the participant through a neurological and educational evaluative process that evaluates the integrity of the sensory and motor systems that will be selected for stimulation and the development of a personalized program comprising the steps of:
i. observing and taking a client history to determine behavior patterns and history of complaints
ii. testing the visual field
iii. audiological testing
iv. neurological examining of:
1. visual system,
2. all reflexes,
3. coordination,
4. musclular skeletal system, and
5. sensory systems
v. quantifying the data and findings into a decision tree for parameters and sensory stimulation selection;
placing the participant on a dual motion platform that provides:
operator seats
a primary master control system,
a secondary control system and
a motion controller interface station
positioning the participant on the support platform in a lying or seated position;
selecting appropriate stimulation based on the above assessing, from a group of stimulations including:
a sensory tactile stimulation to the participant;
a viewing optical light instrument in front of the participant for a visual sensory input;
an auditory sensory input source, such as a headphone, or an external speaker as an auditory sensory input means;
a vasopneumatic pump sleeve on a selected extremity of choice as a means for a compression sensory input
a neuromuscular stimulation pad on the participant as a means of neuromuscular sensory stimulation input
an audio-visual entrainment device which will provide a neurofeedback entrainment sensory input;
a bio-feedback electrophysiological measurement to provide a data input as a means of manual sensory input control
monitoring bio-feedback and controlling sensory input accordingly.

34. The method of claim 33 wherein the quantifying step for the behavior patterns and history determines a generalized theoretical location of central nervous system imbalance and cerebral cortex lobe imbalance.

35. The method of claim 33 wherein the quantifying step for the visual field test compromises campimetery and/or visual light reflex testing, shape and size of visual field and blind spot.

36. The method of claim 33 wherein the quantifying step is a pure tone threshold test mapping the left and right ears frequency response.

37. The method of claim 33 wherein the quantifying step for the neurological exam comprises determining which cerebral hemisphere, brain lobe, brainstem, cerebellar, central nervous system or peripheral nervous system structures are theoretically dysfunctional.

38. The method of claim 33 wherein the quantifying step takes the objective findings of our assessment and determine the initial settings of the sensory stimulation parameters will be used on said multisensory stimulation apparatus and define the settings for a auditory stimulus means, a viewing optical light instrument visual stimulus means, a neuromuscular stimulation means and a support platform means, a dual motion platform vestibular input means that is controlled through a primary master control operating system or a secondary control operating system providing a means for control of cross modal pairing in very precise timing sequences of said stimulus means based on dual motion platform position and visual and auditory stimulus pairing.

39. The method of claim 37 further comprising the steps of repeating the observing, measuring, quantifying and parameter selection process over time.

40. The multisensory stimulation system according to claim 3 compromising a timed coordination program using auditory and visual cues and response recording software.

41. The multisensory stimulation apparatus of claim 3 compromising a means of audio-visual entrainment stimulation that is applied by means of input to the viewing optical light instrument and sound input through the headphone jack, the entrainment system will be monitored by means of bio-feedback control unit and allow for the bio-feedback sensor input to the motion controller for 3 axes of motion control and instruction.
